(19) United States
(12) Patent Application Publication
(10) Pub. No.: US 2012/0209319 A1
(43) Pub. Date: Aug. 16, 2012

(54) REINFORCED SURGICAL ADHESIVES AND SEALANTS AND THEIR IN-SITU APPLICATION

(75) Inventors: Havazelet BIANCO-PELED, Haifa (IL); Ohad KIMHI, Modiin (IL)

(73) Assignee: TECHNION-RESEARCH AND DEVELOPMENT FOUNDATION LTD., Haifa (IL)

(21) Appl. No.: 13/327,268

(22) Filed: Dec. 15, 2011

Related U.S. Application Data
(63) Continuation-in-part of application No. PCT/IL2010/000471, filed on Jun. 15, 2010.
(60) Provisional application No. 61/186,911, filed on Jun. 15, 2009.

(51) Int. Cl. A61B 17/03

(52) U.S. Cl. 606/213

(57) ABSTRACT

In situ application of reinforced adhesive: applying uncured and curable matter to a surface, applying biocompatible inert reinforcing agent comprising at least one curing agent to the uncured composition; allowing curing within subject, cured composition together with the added reinforcing agent being configured to have improved mechanical support and strength.

### FIELD OF THE INVENTION

The present invention relates to surgical adhesives and sealants. More particularly, the present invention relates to reinforced surgical sealants, methods of producing them and methods of their in-situ application.

### BACKGROUND OF THE INVENTION

Surgical adhesives have been increasingly used to enhance or at least partially replace traditional wound closure technologies such as sutures and staples, offering improved sealing capabilities and plugging of undesired leaks.

Despite recent developments and increased clinical demand, most of the currently available products still suffer from serious drawbacks. One of the drawbacks of some available surgical adhesives and sealants is their relatively poor mechanical and tissue-bonding properties. Another drawback of some of the available surgical adhesives is the short time-window available for their proper application onto the treated site.

For example, in dual-component systems, upon mixing of the two components, prior to application onto the treated site, a curing (or cross-linking) process begins. The commercially available Bioglue (http://www.cryolife.com/products/bioglue-surgical-adhesive) is an exemplary system, in which a cross-linker and cross-linkable material glutaraldehyde and BSA respectively may be used for example in the fixation of surgical meshes in hernia repair; cross-linking starts in the applicator.

In general, the process usually takes between a few seconds and a few minutes before the liquid adhesive solidifies. The medical professional has to apply the adhesive onto the desired site quickly, before it completely cures. The short time window might pose many obstacles, such as uniformly spreading the adhesive, completely covering a desired area with an adhesive layer, and completing the application of the adhesive before the adhesive applicator jams with cured adhesive, for example.

Some surgical adhesives may be capable of working around the limitation of curing outside the body, by allowing at least part of the curing to occur after or during application of components of the adhesive to a desired surface, such as an internal organ.

US20010080838 to TYCO HEALTHCARE [US] describes biocompatible implants including a polymer substrate and a reactive component for implant fixation in situ. The reactive component in combination with the substrate is supposed to create a reactive implant which can bond to a tissue surface in situ.

The substrate, for example a mesh, is described therein as being itself reactive with the reactive component.

Similarly, EP0610056 to Johnson and Johnson describes a hydrogel dressing suitable for use in a pressure sensitive adhesive bandage that includes a hydrogel made of a crosslinkable polymer and water and in face-to-face relationship with a substrate having anchoring projections extending into said hydrogel layer such that the hydrogel is anchored to said substrate.

US2002122944 to ST JUDE MEDICAL INCORPORATED [US] adhesive compositions are described as useful in surgical settings such as implanting bioprostheses and/or in manufacturing of bioprostheses, wherein biocompatible substrates can be adhered together by an adhesive bond formed by curing of the epoxyamine adhesive composition.

Notably, the compositions described therein include two substrates bonded to each other.

US2005069589 to Lowinger et al. [US] describes a tissue adhesive sealant that includes a cross-linkable protein in a solution, that when combined with a cross-linking agent solution including an aldehyde and amino acid containing species reactive with the aldehyde, cross-links to form a seal. The sealant is stated to be highly suitable for bonding tissue alone or in combination with a patch.

The patch is described as being brought into contact with the tissue. According to the statement of Lowinger et al., the sealant works fairly equally well with and without the patch, and the patch-sealant may be provided as a unit and/or simultaneously administered.

WO09153751 to LIFEBOND LTD [IL] is directed to a method for cross-linking albumin for use as a sealant or glue for a biological system, for example to induce hemostasis. The cross-linked albumin is stated to be optionally applied as part of a bandage.

One described option for application of the glue or sealant is application of the cross-linkable albumin and cross-linker together with a backing or bandage, or absorbing, adsorbing, combining or otherwise adhering the components to the backing or bandage.

It is apparent that the components are added to the backing or bandage, which is only then introduced into the biological system.

Similarly, in WO10059280 to E I DU PONT DE NEMOURS AND COMPANY, a fibrous tissue sealant in the form of an anhydrous fibrous sheet is described, the sealant comprising a first component which is a fibrous polymer containing electrophilic or nucleophilic groups and a second component capable of crosslinking the first component, the two components supposedly being together when the sheet is exposed to an aqueous medium, thereby immediately forming a crosslinked hydrogel that is adhesive to biological tissue.

Also similarly, AU199923147 to DAVID A BROWDIE [US] describes a tissue adhesive for controlling vigorously bleeding tissues comprising: a mixture of ultrasonically treated fibrous protein, ultrasonically treated globular protein, a cross-linking agent, and a bio-compatible tissue patch.

The patch described used in experiments described therein is coated with the tissue adhesive, and thus all the components including the patch are concomitantly applied to the tissues.

Such systems may not be easy to use as they may limit the time-window for application of the adhesive, and they also may not be optimized for maximal mechanical strength of the resultant constructs, or the adhesion strength of the adhesive.

One object of the present invention is to provide improved and simple reinforced compositions and methods of improved application of surgical adhesives and sealants that allow maximum control of the time of application of the adhesive.

### SUMMARY OF THE INVENTION

According to one aspect of the invention, a multi component reinforced adhesive is provided, the adhesive comprising:
i. an uncured and curable spreadable composition of matter excluding epoxyamine, and water miscible cross-linkable polymers from a group consisting of: naturally existing form of a carbohydrates, synthetically prepared form of carbohydrates, salts of polysaccharides, and
ii. a biocompatible inert reinforcing agent comprising at least one curing agent or water soluble salt thereof,

wherein the uncured composition applied to a surface is characterized as capable of curing after adding the reinforcing agent to the uncured composition, wherein the cured composition together with the added reinforcing agent is configured to have improved mechanical support and strength.

Preferably, the reinforcing agent is configured to provide mechanical support and strength to the adhesive.

The curable composition is typically cross-linkable or polymerisable and the curing agent is typically capable of curing the uncured composition.

In some embodiments, the reinforcing agent comprises a thin film selected from one or more of a group of supports comprising plastic, knitted mesh of fabric made from synthetic or natural polymer, and gauze, made of at least one of the materials selected from the group comprising: oxidized regenerated cellulose, and a hydrophilic water-soluble polymer selected from one of more of: polyethylene glycol, polyvinyl alcohol, alginate, collagen and dextran, and combinations thereof.

In some embodiments, the uncured composition comprises at least one protein selected from a group comprising: albumin, collagen, gelatin, fibrin, fibrinogen, and combinations thereof.

In some of the embodiments in which the uncured composition comprises proteins, the uncured composition further comprises factor XIII.

The curing agent for the proteins may for example be at least one agent selected from a group comprising: glutaraldehyde, formaldehyde, transglutaminase, and thrombin, wherein the curing agent depends upon the curable composition that is selected.

The adhesive including proteins may further comprise calcium ions.

As an example, the adhesive may include a curable composition comprising an albumin-based fluidic solution and the curing agent may comprise an aldehyde selected from one or more of a group comprising: glutaraldehyde and formaldehyde.

As another example, the curable composition may comprise a gelatin-based fluidic solution and the curing agent may comprise transglutaminase.

In some other embodiments, the curable material comprises at least one water-miscible synthetic composition selected from a group comprising: cyanoacrylates, PEG-diacrylates, materials carrying at least two double bonds and mixtures of cross-linkable materials and linear polymer/s and/or monomers.

For example, the curing agent for such curable materials may comprise a photopolymerization initiator such as 1-Hydroxycyclohexyl phenyl ketone.

The uncured compositions applied to a surface may be capable of increasingly stiffening, after adding the reinforcing agent comprising at least one curing agent to the uncured composition.

According to another aspect of the invention, a multi component reinforced adhesive is provided, the adhesive comprising:

A mixture comprising at least one curable protein selected from a group comprising: albumin, collagen, gelatin, fibrin and fibrinogen and combinations thereof, optionally in combination with factor XIII, and one or more curing agent selected from the group comprising: glutaraldehyde, formaldehyde, transglutaminase and thrombin optionally in combination with calcium ions, the cross-linking agent being dependent on the selected curable protein, and
a biocompatible inert reinforcing agent,
wherein the cured mixture together with the added reinforcing agent is configured to have improved mechanical support and strength.

According to a similar aspect, the reinforced multicomponent adhesive comprises:

A mixture comprising at least one curable water-miscible synthetic polymer selected from a group comprising: cyanoacrylates, PEG-diacrylates, and polymers carrying at least two double bonds and a mixture of a linear polymer/s and a cross-linkable polymer, and one or more curing agent comprising a photopolymerization initiator, and
a biocompatible inert reinforcing agent,
wherein the cured mixture together with the added reinforcing agent is configured to have improved mechanical support and strength.

The photopolymerization initiator may comprise 1-Hydroxycyclohexyl phenyl ketone.

According to yet another similar aspect,

A reinforced multicomponent adhesive is provided that comprises:
A mixture comprising bovine serum albumin (BSA) and glutaraldehyde, and
a biocompatible inert reinforcing agent,
wherein the cured mixture together with the added reinforcing agent is configured to have improved mechanical support and strength.

In various embodiments, the reinforcing agent may be placed within or on top of the mixture before or while the mixture is increasingly adhering to the surface.

Preferably, the reinforcing agent is configured to provide mechanical support and strength to the adhesive.

The reinforcing agent may comprise a thin film selected from one or more of a group of supports comprising plastic, knitted mesh of fabric made from synthetic or natural polymer, and gauze, made of at least one of the materials selected from the group comprising: oxidized regenerated cellulose, and a hydrophilic water-soluble polymer selected from one of more of: polyethylene glycol, polyvinyl alcohol, alginate, collagen and dextran, and combinations thereof.

In some embodiments, the mixture further comprises non-soluble suspended solids.

In some embodiments, the adhesive is further characterized by the mixture applied to a surface being capable of increasingly stiffening after adding the curing agent to the mixture.

Any of the embodiments described above may further comprise one or more therapeutic materials selected from one or more of a group comprising drugs, therapeutic proteins, growth factors, and hormones.

According to another aspect, a method of in situ application of a reinforced adhesive to a subject is provided, the method comprising:

applying an uncured and curable composition of matter excluding, epoxyamine and water miscible cross-linkable polymers from a group consisting of: naturally existing form of a carbohydrates, synthetically prepared form of carbohydrates and salts of polysaccharides to a surface,
applying a biocompatible inert reinforcing agent comprising at least one curing agent or water soluble salt thereof to the uncured composition;
allowing curing within the subject, the cured composition together with the added reinforcing agent being configured to have improved mechanical support and strength.

Said surface may be selected from a group comprising tissue surface, synthetic graft surface, and organ surface for example.

Said application of adhesive may comprise sealing or closing an opening in the surface.

The sealing may be of suture lines in tissues, organs, synthetic grafts or combinations thereof for example.

The method preferably further comprises allowing said uncured composition applied to the surface to cure after adding the reinforcing agent to the uncured composition.

According to another aspect, use of the reinforced adhesives for sealing or closing an opening in a surface selected from a group comprising tissue surface, synthetic graft surface, and organ surface is provided.

More specifically, use of the reinforced adhesives for sealing suture lines in tissues, organs, synthetic grafts or combinations thereof is provided.

The use may further comprise treating a subject with one or more therapeutic material selected from one or more of a group comprising drugs, therapeutic proteins, growth factors, and hormones, wherein the adhesive further comprises the one or more therapeutic materials.

It should be noted that the term “adhesive” is used hereinbelow describe materials capable of adhering to surfaces. The particular term “sealant” is defined as materials capable of adhering to a surface while preventing fluid leaks from the surface.

The term “curing” is defined as a process of linking between particles within a curable composition that results in hardening of the composition, during which the composition gains strength.

The term “cross-linking” is defined as forming bonds that link one polymer to other polymers. The bonds may be covalent bonds or physical bonds such as ionic bonds. The Polymers may be either synthetic polymers or natural polymers of mixtures thereof.

### BRIEF DESCRIPTION OF THE FIGURES

For a better understanding of the invention and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments of the present invention only, and are presented in the cause of providing what is believed to be the most useful and readily understood description of the principles and conceptual aspects of embodiments of the invention. In this regard, no attempt is made to show structural details in more detail than is necessary for a fundamental understanding of the embodiments; the description taken with the drawings making apparent to those skilled in the art how the several forms of the invention may be embodied in practice. In the accompanying drawings:

FIG. 1 shows a reinforced sealant being placed on a tissue

FIG. 2 depicts a graph comparing the successful sealing of blood vessels with a reinforced adhesive to the sealing with the same adhesive but without reinforcement.

### DETAILED DESCRIPTION OF THE INVENTION

It is an object of the present invention to provide a reinforced surgical adhesive and a method of applying the same. The surgical adhesive may be adapted for internal use and be capable of adhering to wet as well as dry surfaces and tissues.

Another object is to provide compositions and methods of applying surgical adhesives and sealants that allow maximum control of the time of application of the adhesive.

It is another object to provide a biocompatible adhesive.

It has now been found that simply placing a reinforcing agent in a curable material or a curable material in a reinforcing agent, before the curable material is cured, may reinforce the curable material to a surprising extent after it is cured.

Furthermore, the combinations of a reinforcing agent, curable material and curing material as described below may each be a preferred way to cure the particular curable material and reinforcing the cured material.

In some embodiments a surgical adhesive includes two separate components:

A viscous spreadable solution, which is a curable composition, for example a solution of a curable e.g. cross-linkable compound in water (the adhesive component), and
an inert reinforcing agent, for example a solid mesh, containing a curing agent. The curing may be capable of causing the curable composition to cure, e.g. by cross-linking the curable material.

Either of the components may also contain other ingredients such as pharmaceutical agents, antioxidants, preservatives, colorants, or others.

In some embodiments the above mentioned components are used in two steps:

Applying e.g. spreading the curable composition on a tissue or organ or synthetic tissue or graft or a combination thereof, and Applying the reinforcing and curing agents on/into the curable composition.

The curing process typically immediately commences and is essentially complete after a while.

It is stressed that in some embodiments the curable compositions may be partially cured before the reinforcing agent is brought into contact with it. However, such compositions still have sufficient remaining curability to allow the reinforcing agent to significantly augment the strength of the construct.

The purpose of use of the above mentioned components is thus one or more of the following:

As a surgical sealant, to prevent or reduce fluid \ gas leak from tissues \ grafts \ tissue-graft combinations; Drug-releasing adhesive for local administration of drugs;
To allow maximum control of the time of application of the adhesive.
Mechanically strengthen the adhesive.
Other purposes.

It will occur that some embodiments include adhesive components well known in the art, and that in particular reinforcing agents have been described as being used in conjunction with some adhesive components. However, small changes in the method of applying the agents and compositions and in the resultant structure of the construct may have a very significant influence on the ease and simplicity of use of the adhesive, as well as the resultant physical properties.

According to another aspect, a curing agent such as a cross-linking agent is embedded within the reinforcing agent. The reinforcing agent is preferably a biocompatible and or biodegradable and or a bioabsorbable fibrous mesh or patch or another fibrous structure, which is used in combination with certain components of a surgical sealant

the film may have a thickness of between 50 μm and 10 mm. Preferably, the thickness is between 100 μm and 5 mm.

More particularly, a liquid component of a surgical sealant may be applied on the treated site, and then the reinforcing agent, preferably in the shape of a fibrous structure containing the curing agent, is placed on top of the liquid, semi-liquid, or pre-gelled component. Following placement of the fibrous component containing the curing-agent, curing process begins spontaneously. This composition allows control of the time of initiation of the curing process, thus eliminating potential problems and inconvenience resulting from rapid and inappropriate application. The fibrous component also provides reinforcement and enhances the mechanical strength of the adhesive.

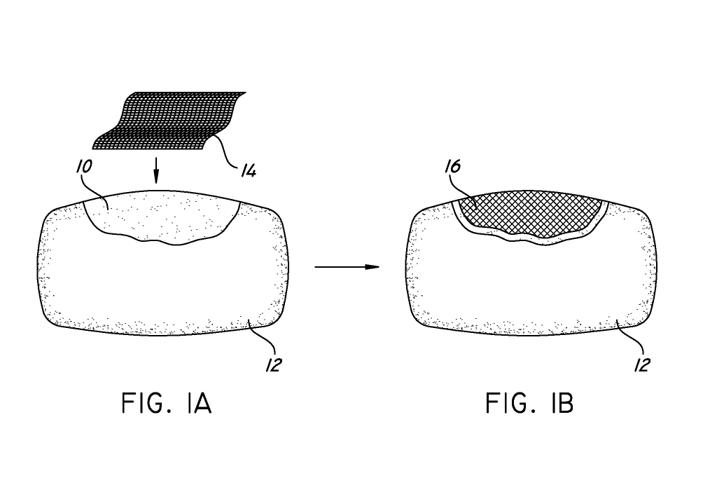

Reference is made to FIGS. 1A and 1B, schematically illustrating a reinforced sealant placed on a tissue in accordance with a preferred embodiment of the present invention. As seen in FIG. 1A, in order to, for example, secure an injured site in a tissue, or seal fluid leaks from injured tissue, such as a blood vessel, intestine, lung or any other organ, a solution 10 made of a cross-linkable material as will be explained herein after, is placed onto an injured tissue 12. An inert reinforcing agent 14 that is made preferably of a solid fibrous mesh type material is placed onto solution 10 or embedded in it. Upon curing, the fibrous solid is incorporated within the cured solution and provides mechanical support and strength to the sealant.

In FIG. 1B, the reinforcing agent is shown incorporated within solution 10 of FIG. 1A. Optionally, a cross linking agent that is preferably already within the reinforcing agent, prior to its placement, is released from the mesh so as to cure solution 10. The solution is cured and the reinforcing agent is incorporated within the cured solution and provides mechanical support and strength to the solution to form a reinforced adhesive 16.

In theory, the curable solution can be placed on the surface (e.g. tissue) before or after the reinforcing agent is introduced; or some of the solution can be placed on the tissue before the reinforcing agent is placed and then another portion is placed in or on the reinforcing agent. However, we believe that in general best results are obtained by first placing the curable composition on the surface, followed by placement of the reinforcing agent on or preferably into the applied curable composition. The curing agent can be applied either before the reinforcing agent is applied, typically just beforehand, or at the same time, preferably by incorporating the curing agent within the reinforcing agent or after the reinforcing agent is applied. We believe that in general it is easier to apply the curing agent either before or during application of the reinforcing agent.

It is stressed that the reinforcing agent in aforementioned embodiments may contact the surface; however, the reinforcing agent according to the present invention does not have a significant role, if any, in the adhesion of the adhesive to the surface, or to the curing process itself.

The curing agent can be added to the curable composition from an outer source after the reinforcing agent is already added to e.g. embedded in the composition, and not from the reinforcing agent itself as mentioned herein before.

In accordance with one embodiment the surgical adhesive comprises:

A solution of cross-linkable material that can be chosen of protein/s, such as albumin, collagen, gelatin, fibrin or fibrinogen optionally in combination with factor XIII, or any other protein. The solution may be lightly cross linked, yet it is preferably still fluidic. A curing agent, such as a cross-linking agent capable of cross-linking the protein/s in the liquid solution described in paragraph 1 above. Such cross-linking agents can be, for example, glutaraldehyde, formaldehyde, transglutaminase, thrombin optionally in combination with calcium ions, or other. The cross-linking agent is dependent on the type of cross-linkable material that is chosen for the adhesive. For example, an albumin-based fluidic solution can be cross linked with glutaraldehyde or formaldehyde; a gelatin-based fluidic solution can be cross linked with the enzyme transglutaminase.

A solid fibrous mesh (reinforcing agent) that can optionally be a patch or another fibrous structure that is preferably biodegradable or bioabsorbable. For example the fibrous structure can be made of either oxidized regenerated cellulose, hydrophilic water-soluble polymer such as polyethylene glycol, polyvinyl alcohol, alginate, collagen, dextran or other material/s. The fibrous structure provides mechanical support to the composition, or enhances its properties in other ways.

The solution of the cross-linkable material and the cross-linking agent can be optionally pre-mixed and placed on the tissue; the reinforcing agent in the shape of solid fibrous mesh is embedded in this solution mixture or placed onto it, preferably prior or close to curing. Upon curing, the fibrous solid is incorporated within the cured polymer or biopolymer and provides mechanical support and strength to the adhesive.

The cross-linkable material, or in more general terms the curable material, spread on tissue for example, will increasingly adhere to the tissue and preferably stiffen also, upon adding to it the reinforcing agent. By “dependent” it is meant that the curing agent is selected according to the particular curable composition, i.e. the curing agent is capable of curing the curable composition. In addition, the curing agent may be selected according to the desired characteristics of the adhesive, for example adhesion strength, rate of curing, the stiffness of the adhesive and its mechanical strength.

According to another embodiment, the surgical adhesive comprises:

A solution of synthetic cross-linkable materials, such as compositions selected from cyanoacrylates, PEG-diacrylates, or other synthetic materials carrying at least two double bonds. Alternatively, the solution may contain a mixture of cross-linkable materials and linear polymer/s or monomers. The solution may be lightly cross linked, yet it is preferably still fluidic.
An appropriate initiator that is capable of inducing cross-linking of the cross-linkable material present in the liquid solution and described in paragraph 1 above. Such initiator can be, for example, a photopolymerization initiator such as Irgacure®184 (1-Hydroxycyclohexyl phenyl ketone) that initiates a chemical reaction upon irradiation with light.
A reinforcing agent that can be a solid fibrous mesh; optionally a patch or another fibrous structure. For example the fibrous structure can be made of either oxidized regenerated cellulose, hydrophilic water-soluble polymer such as polyethylene glycol, polyvinyl alcohol, alginate, collagen, dextran or other material/s. The fibrous structure provides mechanical support to the composition, or enhances its properties in other ways.

The solution of the cross-linkable material and the initiator are pre-mixed and placed onto the tissue; the solid fibrous mesh is embedded within the solution or placed onto it, preferably prior or close to curing. Upon curing, the fibrous solid is incorporated within the solution and provides mechanical strength to the adhesive.

According to a yet another embodiment, the surgical adhesive comprises:

A solution of cross-linkable material that can be chosen of protein/s, such as albumin, collagen, gelatin, fibrin or fibrinogen optionally in combination with factor XIII, or any other protein. The solution may be lightly cross linked, yet it is preferably still fluidic.
A reinforcing agent that also functions as a solid fibrous carrier of a curing-agent, such as a cross-linking agent. The solid carrier can be a mesh that can optionally be a patch or another fibrous structure that is preferably biodegradable or bioabsorbable. For example the solid carrier can be made of either oxidized regenerated cellulose, hydrophilic water-soluble polymer such as polyethylene glycol, polyvinyl alcohol, alginate, collagen, dextran or other material/s. The curing agent is capable of cross-linking the cross-linkable material present in the liquid solution described in paragraph 1 above. Such cross-linking agents can be, for example, glutaraldehyde, formaldehyde, transglutaminase, thrombin optionally in combination with calcium ions, or other. The cross-linking agent is dependent on the type of cross-linkable material that is chosen for the adhesive. For example, an albumin-based fluidic solution can be cross linked with glutaraldehyde or formaldehyde; a gelatin-based fluidic solution can be cross linked with the enzyme transglutaminase. The curing agent is incorporated within the solid carrier, and is released from it to the liquid component, thus providing a cured composition. The solid carrier optionally provides mechanical support to the gel, or enhances its properties in other ways.

In addition and in accordance with another embodiment, the surgical adhesive comprises:

A solution of cross-linkable materials such as cyanoacrylates, PEG-diacrylates or other synthetic materials carrying at least two double bonds. Alternatively, the solution may contain a mixture of cross-linkable materials and linear polymer/s and/or monomers. The solution may be lightly cross linked, yet it is preferably still fluidic.
A reinforcing agent that also functions as a solid fibrous carrier of an initiator. The solid carrier can be a mesh that can optionally be a patch or another fibrous structure that is preferably biodegradable or bioabsorbable. For example the solid carrier can be made of either oxidized regenerated cellulose, hydrophilic water-soluble polymer such as polyethylene glycol, polyvinyl alcohol, alginate, collagen, dextran or other. The initiator is capable of cross-linking the polymer/s present in the liquid solution described in paragraph 1 above. Such initiator can be for example, a photopolymerization initiator such as 1-Hydroxycyclohexyl phenyl ketone that initiates a chemical reaction upon irradiation with light the initiator is incorporated within the solid carrier, and is released from it to the liquid component, thus providing a cured composition under appropriate conditions. The solid carrier optionally provides mechanical support to the composition, or enhances its properties in other ways.

According to some embodiments, the liquid, semi-liquid or pre-gelled solution described in paragraph 1 of any of the embodiments above, include two or more materials, selected from any of the groups described herein below:
1. A cross-linkable protein, such as albumin, collagen, gelatin, fibrinogen or fibrin optionally in combination with factor XIII, or other curing agent.
2. A cross-linkable polymer, such as cyanoacrylates, PEG-diacrylates, or other synthetic polymers carrying at least two double bonds or a mixture of a linear polymer/s and cross-linkable polymer/s.
3. A cross-linkable or a linear polysaccharide, such as alginate, chitosan, starch, pectin or others.

### Application Methods

Several methods are used to apply adhesive in the site that requires tissue repair or tissue sealing.

A layer of a liquid curable composition is spread on the surface and the reinforcing agent is embedded in it. The composition is allowed to cure.

A layer of a dry curable composition is placed on the surface and the reinforcing agent placed on top of it. The composition is allowed to cure. Optionally addition of liquids, such as saline, may be provided.

A dry curable composition pre-combined with the reinforcing agent is placed on the surface. The composition is allowed to cure. Optionally addition of liquids, such as saline, may be provided.

In all the above cited examples, once the curable composition is cured, it loses its adherence capability thus acts as a barrier that may prevent post-surgical adhesion.

### Measurement of Sealing Capabilities

Sealing capabilities are evaluated in vitro using a pulsating flow system. The system is composed of computer, controller, pump, pressure transducer and solenoid valves.

As a model to human artery, bovine aortas are chosen.

In experiments made in the system, each aorta was connected to the system by plastic connectors.

Punctures were made in aortas in order to simulate a leak. Different leaks were simulated either by simply puncturing the aortas with a 20 mm 0.5 cc surgical needle, or cutting out holes in the aortas of about 16 mm².

Each leak was characterized for flow rate before and after the application of the sealant, i.e. Q₀ and Q_{f}, respectively, were measured. Sealing ratio (SR) was defined as Sealing ratio=1−Q_{f}/Q₀, e.g. sealing ratio of 1 represents complete sealing.

In order to get proper statistics each experiment was repeated six times or more.

### Example 1

A Bovine Serum Albumin (BSA) solution was applied on a bovine artery in which six proximal holes were made with a needle. An ORC (Oxidized Regenerated Cellulose) mesh soaked with 50 micro-liters of glutaraldehyde solution was placed on top of the BSA solution so as to cover all six holes.

The experiment was replicated six times.

A Sealing Ratio (SR) of 1 (complete sealing) was achieved in all six experiments.

### Example 2

16 mm2 holes were made in bovine arteries. Bioglue®, with or without ORC mesh, was used to seal the holes.

The sealing was tested at various pressures ranging from 70/120 mmHg up to 70/270 mmHg, in seven replicates at each pressure.

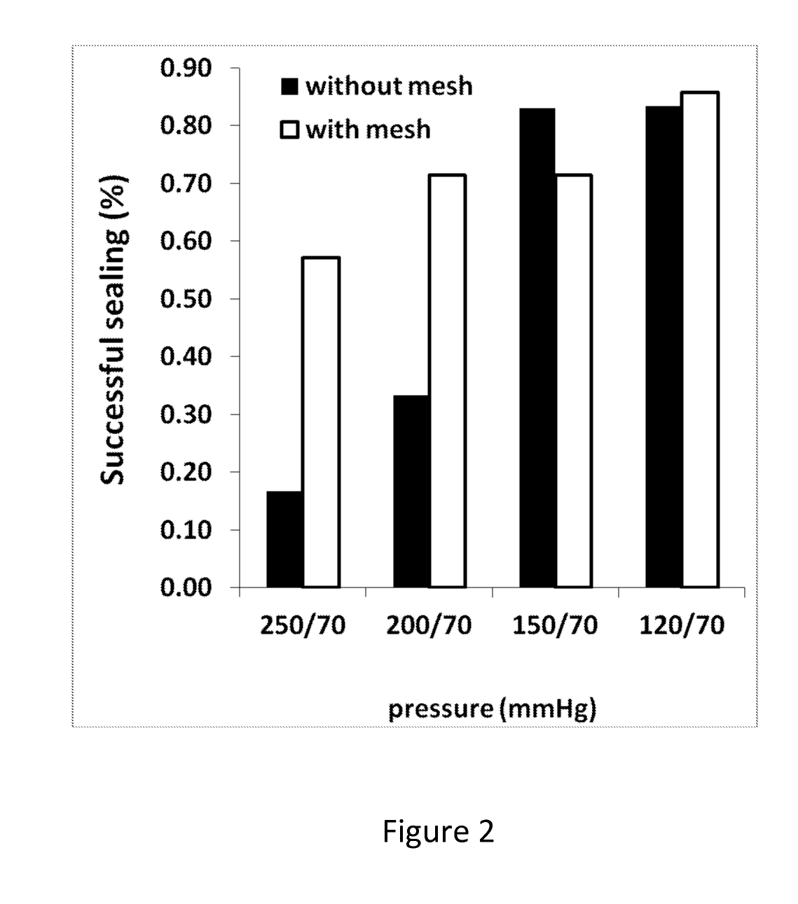

The graph in FIG. 2 shows the percentage of experiments in which the sealing was complete.

It is apparent that in general the reinforcement is more important the higher the pressure in the blood vessel.

### Example 3

A synthetic curable composition polymer, a pre-gel composed of a solution of the cationic hydrophilic polymer poly(allylamine hydrochloride) is spread on a surface. A mesh is soaked in a solution of four- or six-arm PEG succinimidyl glutarate and dried. Upon contact between the mesh and the pre-gel, the PEG diffuses out and a reinforced hydrogel spontaneously forms from the pre-gel and adheres to the surface.

### Example 4

As an example of a protein-based sealant, a pre-gel composed of fibrinogen solution is spread on a surface. A mesh is soaked with a solution containing factor XIII, thrombin and calcium ions, and dried. Upon contact between the mesh and the pre-gel, the factor XIII and thrombin diffuse out of the mesh and crosslink the protein which adheres to the surface.

A synthetic curable composition polymer, a pre-gel composed of a solution of the cationic hydrophilic polymer poly(allylamine hydrochloride) is spread on a surface. A mesh is soaked in a solution of four- or six-arm PEG succinimidyl glutarate and dried. Upon contact between the mesh and the pre-gel, the PEG diffuses out and a reinforced hydrogel spontaneously forms from the pre-gel and adheres to the surface.

A synthetic curable composition polymer, a pre-gel composed of a solution of the cationic hydrophilic polymer poly(allylamine hydrochloride) is spread on a surface. A mesh is soaked in a solution of four- or six-arm PEG succinimidyl glutarate and dried. Upon contact between the mesh and the pre-gel, the PEG diffuses out and a reinforced hydrogel spontaneously forms from the pre-gel and adheres to the surface.

A synthetic curable composition polymer, a pre-gel composed of a solution of the cationic hydrophilic polymer poly(allylamine hydrochloride) is spread on a surface. A mesh is soaked in a solution of four- or six-arm PEG succinimidyl glutarate and dried. Upon contact between the mesh and the pre-gel, the PEG diffuses out and a reinforced hydrogel spontaneously forms from the pre-gel and adheres to the surface.

A synthetic curable composition polymer, a pre-gel composed of a solution of the cationic hydrophilic polymer poly(allylamine hydrochloride) is spread on a surface. A mesh is soaked in a solution of four- or six-arm PEG succinimidyl glutarate and dried. Upon contact between the mesh and the pre-gel, the PEG diffuses out and a reinforced hydrogel spontaneously forms from the pre-gel and adheres to the surface.

The examples described above present various selected embodiments of a multi component adhesive for preparation of adhesives and methods of preparation of the adhesives. It is noted that further embodiments are anticipated which also fall within the scope of the present invention. The scope of the present invention is defined by the claims and includes both combinations and sub combinations of the various features described hereinabove as well as variations and modifications thereof, which would occur to persons skilled in the art upon reading the foregoing description.

In the claims, the word “comprise”, and variations thereof such as “comprises”, “comprising” and the like indicate that the components listed are included, but not generally to the exclusion of other components.

What is claimed:

1. A reinforced multi component adhesive comprising:
a) layer of uncured and curable composition of matter, and
b) a biocompatible inert reinforcing agent comprising at least one curing agent and a thin film selected from one or more of a group of supports comprising plastic, knitted mesh of fabric made from synthetic or natural polymer, and gauze, made of at least one of the materials selected from the group comprising: oxidized regenerated cellulose, and a hydrophilic water-soluble polymer selected from one of more of: polyethylene glycol, polyvinyl alcohol, alginate, collagen and dextran, and combinations thereof,
wherein the uncured composition layer is characterized as capable of curing after adding the reinforcing agent to the uncured composition, and wherein the cured composition together with the added reinforcing agent is configured to have improved mechanical support and strength,
and wherein:
when the uncured composition comprises at least one protein selected from a group comprising: albumin, collagen, gelatin, fibrin, fibrinogen, and combinations thereof, the curing agent depends upon the protein that is selected:
when the curable composition comprises an albumin-based fluidic solution, the curing agent comprises an aldehyde selected from one or more of a group comprising: glutaraldehyde and formaldehyde;
when the curable composition comprises a gelatin-based fluidic solution, the curing agent comprises transglutaminase;
when the curable composition comprises a fibrinogen solution, the curing agent comprises calcium ions, and either the curable composition or the curing agent further comprise factor XIII and thrombin;
when the curable material comprises at least one water-miscible synthetic composition selected from a group comprising: cyanoacrylates, PEG-diacrylates, materials carrying at least two double bonds and mixtures of cross-linkable materials and linear polymer/s and/or monomers, the curing agent comprises a photopolymerization initiator,
and when the curable material comprises cationic hydrophilic polymer poly(allylamine hydrochloride), the curing agent comprises four- or six-arm PEG succinimidyl glutarate.

2. The adhesive of claim 1, wherein the reinforcing agent is configured to provide mechanical support and strength to the adhesive.

3. The adhesive of claim 1, wherein photopolymerization initiator comprises 1-Hydroxycyclohexyl phenyl ketone.

4. The reinforced adhesive of claim 1, wherein the adhesive is further characterized by the uncured composition applied to a surface being capable of increasingly stiffening after adding the reinforcing agent comprising at least one curing agent to the uncured composition.

5. The reinforced adhesive of claim 1, wherein said uncured composition and/or curing agent further comprise non-soluble suspended solids.

6. The reinforced adhesive as claimed in claim 1, further comprising one or more therapeutic materials selected from one or more of a group comprising drugs, therapeutic proteins, growth factors, and hormones.

7. A method of in situ application of a reinforced adhesive to a subject comprising:
applying an uncured and curable composition of matter excluding, epoxyamine and water miscible cross-linkable polymers from a group consisting of: naturally existing form of carbohydrates, synthetically prepared form of carbohydrates and salts of polysaccharides to a surface,
applying a biocompatible inert reinforcing agent comprising at least one curing agent to the uncured composition;
allowing curing within the subject,
the cured composition together with the added reinforcing agent being configured to have improved mechanical support and strength.

8. The method as claimed in claim 7, wherein said surface is selected from a group comprising tissue surface, synthetic graft surface, and organ surface.

9. The method as claimed in claim 7, wherein said application of adhesive comprises sealing or closing an opening in the surface.

10. The method as claimed in claim 7, further comprising sealing suture lines in tissues, organs, synthetic grafts or combinations thereof.

11. The method of claim 7, further comprising allowing said uncured composition applied to the surface to cure after adding the reinforcing agent to the uncured composition.

12. The reinforced multi component adhesive of claim 1, wherein the film has a thickness of between 50 μm and 10 mm.

13. The adhesive of claim 12, wherein the thickness is between 100 μm and 5 mm.
